(19) United States
(12) Patent Application Publication
(10) Pub. No.: US 2007/0135796 A1
(43) Pub. Date: Jun. 14, 2007

(54) Method and apparatus for applying medication to internal tissue

(76) Inventor: George Gorodeski, Beachwood, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/576,214

(22) PCT Filed: Oct. 21, 2004
(86) PCT No.: PCT/US04/34782

§ 371 Date: Apr. 17, 2006

Related U.S. Application Data
(60) Provisional application No. 60/513,463, filed on Oct. 22, 2003.

(51) Int. Cl. A61K 9/22

(52) U.S. Cl. 604/892.1

(57) ABSTRACT

A method and apparatus for applying medication to internal human tissue. One drug delivery member (24) for applying medication (20) to internal human tissue includes a medication impermeable barrier (26) and a medication-carrying matrix (28). The medication impermeable barrier is shaped to conform to a contour of the internal tissue. The medication-carrying matrix releases the medication to the human tissue upon being applied to the internal human tissue. The matrix may be adapted to adhere to the internal human tissue.

### RELATE BACK

The present application claims priority from provisional application Ser. No. 60/513,463, filed on Oct. 22, 2003, entitled “A Novel Method for Treatment Of Cervical Dysplasia in Women,” which is incorporated herein by reference in its entirety.

### FIELD OF THE INVENTION

The present application relates to techniques for applying medication to internal human tissue and, more particularly, the present application relates to techniques that localize the applied medication

### BACKGROUND OF THE INVENTION

Internal organ tissues can be adversely affected by a variety of diseases. Some disease conditions can become chronic, and if not treated can lead to morbidity and mortality. Advancements in diagnostic modalities allow earlier detection of chronic disease such as cancer. Cancers can now be diagnosed at early stages of lesion development, including pre-invasive lesions, whereby the disease is localized to a relatively small area of tissue. In the USA about 13,000 women are diagnosed annually with invasive uterine cervical cancer, and 4,500 women die annually of cervical cancer.

The human uterine cervix is a cylindrical shaped organ; its lower portion (ectocervix) opens into the vagina, is lined with squamous epithelial cells and is visible with the use of a speculum. The ectocervix connects with the main part of the uterus through an inner part (endocervix), having a cervical canal continuous with the uterine cavity that is lined with columnar epithelium cells. The ectocervical and endocervical epithelial cells intersect at a region called the squamo-columnar junction that is usually located at the upper/inner part of the ectocervix.

Almost all cases of invasive cervical cancer arise out of a region of cervical dysplasia. Cervical dysplasia is an area on the cervix where cells have undergone abnormal changes and have transformed into a precancerous lesion. Cervical dysplasias most commonly develop at the squamo-columnar junction of the cervix. The lesions usually grow focally and develop into severe dysplasia, localized cancer, and invasive cancer. Such lesions are described collectively as cervical intraepithelial neoplasia (CIN), a range of epithelial abnormalities ranging from mild (CIN I), moderate (CIN II) to severe (CIN III).

In the USA about one of every 10-20 women will be diagnosed with cervical dysplasia during ages 18 to 55. The current standard-of-care advocates follow-up of mild cases of dysplasia, and surgical treatment of severe cases of dysplasia This entails little, or no action in mild cases, versus aggressive management in severe cases. Lack of a safe, effective and cost-effective treatment modality of cervical dysplasia is considered one of the reasons for the continued increase in invasive cervical cancer, and it contributes to escalating medical costs associated with the disease (estimated at $6 billion annually).

Dysplastic cervical cells are rapidly proliferating cells that can be growth arrested and destroyed by a number of known drugs. However, previous drug treatments for cervical dysplasia were unsuccessful because of low benefit—risk considerations: to effectively destroy the small foci of dysplasia on the cervix, a systemic (general) administration of a high dose of the drug was necessary to effectively destroy the small foci of dysplasia on the cervix. This resulted in systemic toxic side effects out of proportion for the desired local effect.

Drug treatments for cervical dysplasia can be also effective when applied locally to the lesions, thus providing an advantage over systemic drug administration that can produce systemic side effects. However, previous localized drug treatments for cervical dysplasia were unsuccessful because of difficulties in the application and retention of drugs onto the cervix, and because the drugs tend to migrate and cause side effects to healthy tissue.

### SUMMARY

Earlier detection of chronic diseases, such as cancer, provides the opportunity for local treatment and eradication of the disease. The main advantage in this type of management is that treatment, such as drugs, can be provided or applied locally at the site of the lesion, while at the same time surrounding tissues or more distant tissues are not exposed to the effect of the strong drugs.

The present application describes a novel method and apparatus for the treatment of diseases localized to body surfaces. However, the disclosed method and apparatus could be employed to treat localized disease anywhere in the body.

The present application concerns a methods and devices for applying medication to internal human tissue. In one embodiment, the device is a drug delivery member or patch for applying medication to internal human tissue that includes a medication impermeable barrier and a medication carrying matrix. The medication impermeable barrier conforms to a contour of the internal tissue The medication carrying matrix is disposed on the barrier. The medication carrying matrix releases the medication to the human tissue when the drug delivery member is applied to the internal human tissue. The matrix may be adapted to adhere to the internal human tissue and/or a retainer may be used to secure the drug delivery member to the internal tissue. In one embodiment, the drug delivery member includes a medication-free deposit of matrix near an edge of the barrier. This deposit inhibits migration of the medication past the edge of the barrier.

The application provides an example of the method and device applied to a common disease in women, cervical dysplasia and localized cervical cancer. However, the method and device could be used to apply medication to any internal bodily lesion. In one embodiment, the barrier is formed with an inner surface that corresponds to an outer surface of a patient's cervix. For example, the barrier may include a cap portion that is configured to fit over an outer periphery of the cervix and a projection that is configured to extend into the cervical canal. The projection may be sized to extend into the cervical canal, past a squamo-columnar epithelial junction. The medication may be dispersed in the matrix such that the medication is concentrated in an area surrounding the squamo-columnar epithelial junction when the drug delivery member is applied to a cervix.

A variety of different medications may be carried applied by the drug delivery member. Examples of medications include but are not limited to 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, and subcombinations thereof.

A variety of matrices can be used to carry and release the medication. For example, synthetic and natural carboxy polymers and co-polymers whose fluidity is pH dependent, such as carboxy vinyl polymers and co-polymers whose fluidity is pH dependent can be used as matrices. The barrier may be made from a material that dissolves after the medication is transferred from the matrix to the internal tissue.

In one method of inserting a drug delivery member onto the cervix of a patient, the drug delivery member is mounted on an applicator. The applicator is inserted near the cervix of said patient. The drug delivery member is released from the applicator onto the cervix of the patient. A first portion of the drug delivery member may be placed over an outer periphery of a cervix and a second portion of the drug delivery member may be inserted into the cervical canal, past a squamo-columnar epithelial junction. The medication may be applied to the drug delivery member such that the medication is concentrated in an area surrounding the squamo-columnar epithelial junction.

In one embodiment, the drug delivery includes a retainer for securing the drug delivery member to the cervix. One retainer may be configured to apply pressure to an outer circumferential surface of the cervix and the cervical canal to secure the barrier to the cervix. Such a retainer could take a variety of forms. One example is a retainer that comprises one or more ribs that apply pressure to an outer circumferential surface of the cervix and the cervical canal to secure the barrier to the cervix. Another example is a retainer that comprises an elastic band secured to the cap and a ring secured to the projection that expands upon insertion.

Further advantages and benefits of the invention will become apparent to those skilled in the art after considering the following description and appended claims in conjunction with the accompanying drawings.

### BRIEF DESCRIPTION OF THE FIGURES

FIG. 1 is a side cross-sectional illustration of a drug delivery member applied to internal tissue of a patient;

FIG. 2 is an illustration similar to FIG. 1 that shows medication being delivered from the drug delivery member to the tissue;

FIG. 2A is a side cross-sectional illustration of a drug delivery member applied to internal tissue of a patient;

FIG. 3 is an illustration that shows the drug delivery member removed and the medication delivered to the internal tissue;

FIG. 4 is an anatomical illustration of organs of a female patient;

FIG. 5 is an enlarged view of the portion of FIG. 4 that is labeled FIG. 5;

FIG. 6 is a schematic illustration of a drug delivery member applied to a cervix;

FIG. 7 is a side cross-sectional view of an applicator for inserting a drug delivery member to a cervix of a patient;

FIG. 8A is a side cross-sectional view of the applicator m the folded state of FIG. 1 shown with a drug delivery member mounted thereon just prior to application to the cervix of a patient;

FIG. 8B is a side view of the applicator of FIG. 1 in the unfolded state, shown with a drug delivery member mounted thereon during application to the cervix of a patient;

FIG. 8C is a side view of the applicator of FIG. 1 in the unfolded state and in an inflated state;

FIG. 8D is a side view of the applicator of FIG. 1 separated from a drug delivery member applied to the cervix of a patient;

FIG. 9 is a side cross-sectional view of an applicator for inserting a drug delivery member to a cervix of a patient;

FIG. 10 is an illustration of an applicator for inserting a drag delivery member onto a cervix of a patient;

FIG. 11 is a perspective view of a drug delivery member in the uninstalled state;

FIG. 12 is a perspective view of the drug delivery member of FIG. 11 in the installed state;

FIG. 13 is a cross-sectional view of the drug delivery member of FIGS. 11 and 12 placed over the cervix of a patient;

FIG. 14 is a cross-sectional view of the drug delivery member of FIGS. 11 and 12 secured to a cervix of a patient;

FIG. 15 is a schematic illustration of an applicator having inserted a drug delivery member onto a cervix of a patient; and

FIG. 16 is a schematic illustration of a drug delivery member secured to a cervix of a patient.

### DETAILED DESCRIPTION OF THE INVENTION

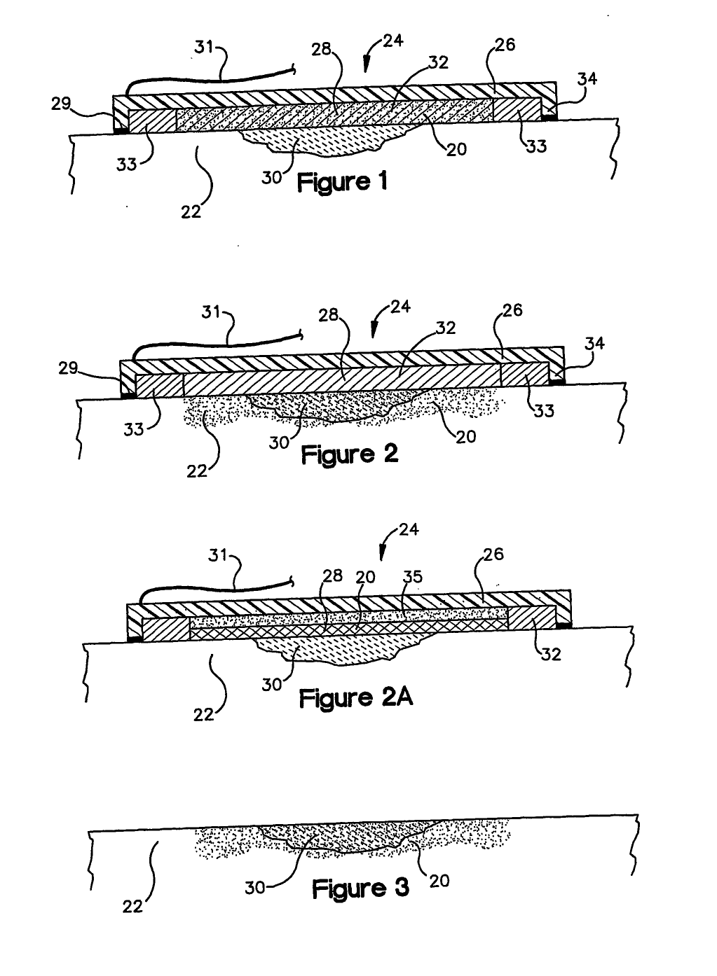

The present application concerns a method and apparatus for applying medication 20 to internal human tissue 22. FIGS. 1-3 illustrate a drug delivery member 24 or patch affixed to internal tissue 22 for applying medication 20 to the tissue. The drug delivery member 24 includes a medication impermeable barrier 26 and a medication carrying matrix 28 disposed on the barrier. In the exemplary embodiment, the barrier is formed from a pliable material that conforms to a surface of the internal human tissue. In the embodiment depicted by FIGS. 1 and 2, the matrix adheres to the tissue 22 and releases the medication to the tissue upon being applied to the tissue.

In the example of FIGS. 1-3, the tissue 22 includes a disease affected area 30. A medicated portion 32 of the matrix is sized to apply medication 20 to the affected area 30 plus a small margin around the affected area Healthy tissue outside the margin is not significantly exposed to the medication 20. In the example of FIGS. 1 and 2, a medication-free deposit 33 of matrix in a region near an edge 34 of the barrier that inhibits migration of the medication past the edge of the barrier.

A variety of matrices can be used to carry and release the medication. In one embodiment, the matrix also secures the drug delivery member 24 to the tissue 22. Examples of acceptable matrices include synthetic and natural carboxy polymer and co-polymers whose fluidity is pH dependent, such as carboxy vinyl polymers and co-polymers whose fluidity is pH dependent can be used as matrices. Additional examples of acceptable matrices include various carbopols, pectin, polyvinylpyrolidon, guar gum, ethylene maleic anhydride resins and/or mixtures thereof. In one embodiment, the matrix has a pH in the range of 2.5 to 7.5.

In the example of FIG. 2A, the drug delivery member 24 further comprises a reservoir 35 disposed between the barrier 26 and the matrix 28 for storing the drug formulation therein. When applied to the patient, the medication 20 passes from the reservoir, though the matrix 28 and to the tissue 22. Such a reservoir would be included when the required amount of medication cannot be dispersed in a suitable matrix 28, or to provide a time delay for the release of medication 20.

Referring to FIG. 1, the drug delivery member 24 may further optionally comprise a layer of pressure sensitive adhesive 29 for adhering to the surface of the tissue. In the example of FIG. 1, the layer of pressure sensitive adhesive 29 is applied to an edge 34. When included, the pressure sensitive adhesive helps to secure the drug delivery member 24 to the tissue and further inhibits migration of the medication past the edge of the drug delivery member. Alternatively, a bio-adhesive may be applied to the drug delivery member prior to application to the internal tissue, or directly onto the internal tissue prior to installation of the drug delivery member. Examples of acceptable matrices include synthetic and natural carboxy polymer and co-polymers whose fluidity is pH dependent, such as carboxy vinyl polymers and co-polymers whose fluidity pH dependent can be used as matrices. One example of a suitable bioadhesive is polycarbophil, sold under the trade name RepHresh Vaginal Gel, (commercially available from Columbia Laboratories, Inc.).

After the medication is released to the tissue, the drug delivery member 24 is removed. In the example illustrated by FIGS. 1 and 2, a string 31 is attached to the barrier for removing the drug delivery member.

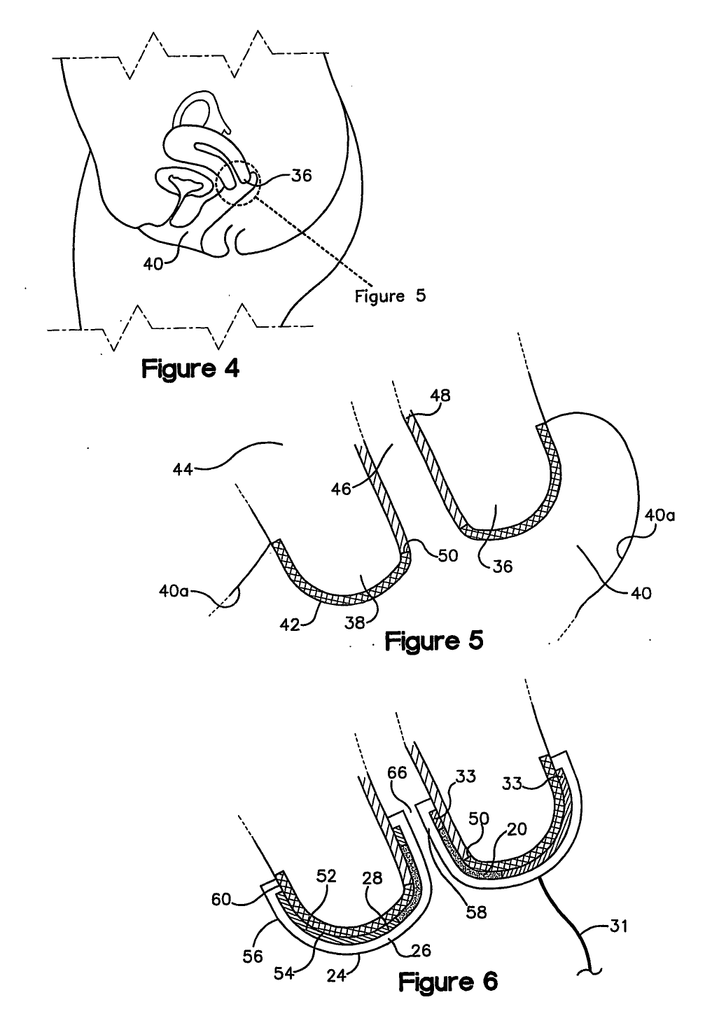

In the exemplary embodiment, the barrier 26 is shaped to conform to a contour of the internal tissue. For example, the barrier 26, and thus the drug delivery member, could be shaped to conform to a surface of a patient's intestinal tract, colon, cervix, or other external and internal organs. FIGS. 4-18 illustrate a cervix and drug delivery members that are shaped to conform to the contour of the cervix.

FIGS. 4 and 5 illustrate a cervix 36. The ectocervix 38 opens into the vagina 40, which is defined by vaginal walls 40a. The ectocervix is lined with squamous epithelial cells 42. The ectocervix 38 connects with the main part of the uterus through the endocervix 44 having a cervical canal 46 continuous with the uterine cavity that is lined with columnar epithelium cells 48. Referring to FIG. 5, the ectocervical and endocervical epithelial cells intersect at the squamo-columnar junction 50.

In the embodiment illustrated by FIG. 6, the barrier 26 is formed with an inner surface 52 that corresponds to an outer surface 54 of the cervix 36. The barrier includes a cap portion 56 and a projection 58. The cap portion 56 that is configured to fit over an outer periphery 60 of the cervix. The projection 58 is radially inward of the cap portion 56 and is configured to extend into the cervical canal 46. The projection 58 extends past the squamo-columnar epithelial junction 50. In one embodiment, the medication 20 is dispersed in the matrix 28 such that the medication is concentrated in an area surrounding the squamo-columnar epithelial junction when the drug delivery member is applied to a cervix.

In the embodiments illustrated by FIGS. 6-16, the drug delivery members 24 are large and flexible enough to conform the matrix to the inner cervical canal and outer region. The center of the drug delivery members illustrated by FIGS. 6, 11-13 and 15-18 include a hole 66 sized sufficiently large enough to allow the passage of fluid. The hole could be omitted if the application time of the drug delivery member is less than 24 hours. In one embodiment, the barrier 26 or outer layer is made from a flexible material. The barrier 26 may comprise any suitable medical grade material that functions to inhibit a substantial amount of the drug 20 from being delivered to the vagina 40. For example, the barrier 26 can be made from medical grade polyvinyl chloride. In one embodiment, the barrier is made from a material that dissolves after the medication is transferred from the matrix to the tissue.

A variety of drugs 20 are suitable for treating cervical intraepithelial neoplasia. Examples of suitable drugs include but are not limited to 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, and subcombinations thereof The therapeutic drugs are present in an amount sufficient to provide a therapeutic effect to a patient.

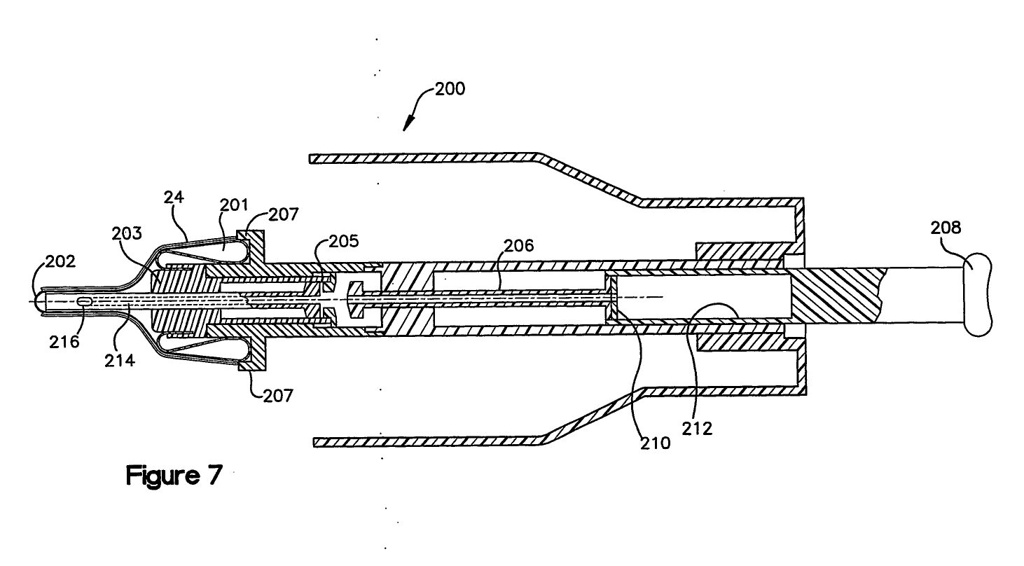

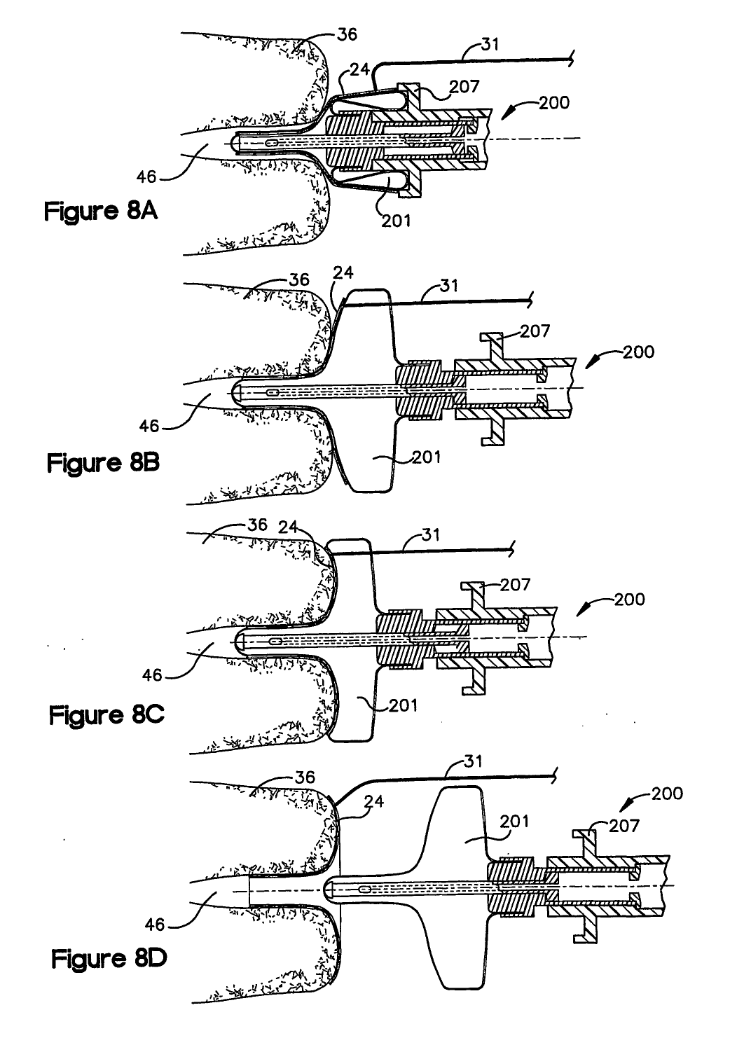

A variety of different applicators could be used to insert the drug delivery member onto the correct location of the patient's cervix. FIGS. 7 and 8A-8D illustrate one tool that could be used as a drug delivery member applicator 200. This tool is described in U.S. Pat. No. 6,475,164 which is hereby incorporated by reference in its entirety. The tool used as a drug delivery member applicator 200 as shown in FIGS. 7 and 8A-8D comprises an inflatable balloon member 201 having an extending tip 202. The inflatable balloon member may be biased into an inflated drug delivery member deployment position as shown in FIGS. 8B-8D or into a deflated folded position as shown in FIGS. 7 and 8A. The inflatable balloon structure is made of a suitable elastomeric material such as rubber, polyurethane, or a thermoplastic elastomer. The drug delivery member 24 is loaded directly onto the folded balloon structure, and together with the balloon is inserted into flange 207 located on the outer portion of the handle.

The balloon member 201 is mounted on sliding member 203 of the handle. A distal end 205 of the sliding member 203 is positioned for contact with end of hollow shaft 206. When button 208 of the handle is depressed, plunger 210 and shaft 206 move in unison until end of shaft makes contact with the sliding member 205. As the sliding member moves relative to the handle, the balloon member 201 and drug delivery member 100 are freed from the retaining flange 207. As the button 208 of the handle is continued to be depressed, the balloon member 201 is filled with air by means of the syringe structure incorporated into the handle. Thus once the end of shaft 206 contacts the sliding member 205, a seal is formed. Continued depression of the button of the handle causes the plunger to move relative to the cylinder barrel 212; forcing air from the cylinder through hollow shaft 206, into hollow stylette 214 and through exit hole 216 of stylette into the interior of the balloon member 201.

In the embodiment illustrated by FIGS. 7 and 8A-8D, the drug delivery member 24 is pre-loaded onto the balloon applicator prior to insertion into a patient. In the exemplary embodiment, the matrix 28 adheres the drug delivery member to the cervix. In an another embodiment, a bioadhesive may be added to the mounting surface prior to installation. The drug delivery member applicator 200 with the balloon member 201 in the folded position is inserted through a speculum into the vagina of a patient. Referring to FIG. 8A, the tip of the applicator 202 is inserted into the cervix 36 until the shoulder of the balloon member is seated against the mouth of the cervix. Referring to FIG. 8B, depressing of the button or trigger at the end of the handle releases the outer ends of the balloon member and drug delivery member from the retaining flange 207. As the balloon member 201 is inflated from the continued depression of the button of the handle, the outer surface of the balloon member presses the drug delivery member against the outer surface of the cervix. In one embodiment, the matrix has bio-adhesive properties that causes the drug delivery member 24 to be retained on the outer surface of the cervix. The applicator balloon may then be deflated by releasing the button of the handle. The applicator may then be removed from the vagina via the speculum. The drug delivery member 24 is left intact upon the patient's cervix until the drug therapy has concluded. The patient or medical personnel may remove the drug delivery member 24 by pulling string 31.

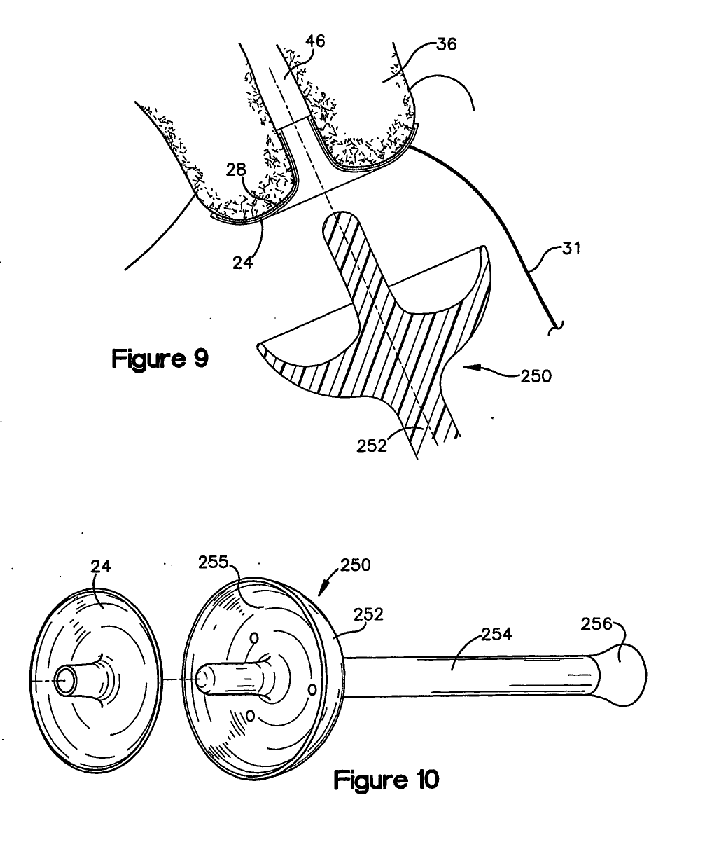

Another example of an applicator 250 is illustrated by FIGS. 9 and 10. The applicator 250 includes a drug delivery member holder 252 and a handle 254 extending from the holder. The holder defines a retaining surface 255 that corresponds to the surface of the cervix. A release mechanism 256 may be included for releasing the drug delivery member 24 onto the cervix. In one embodiment, the release mechanism forces air to the holder that separates the drug delivery member from the holder.

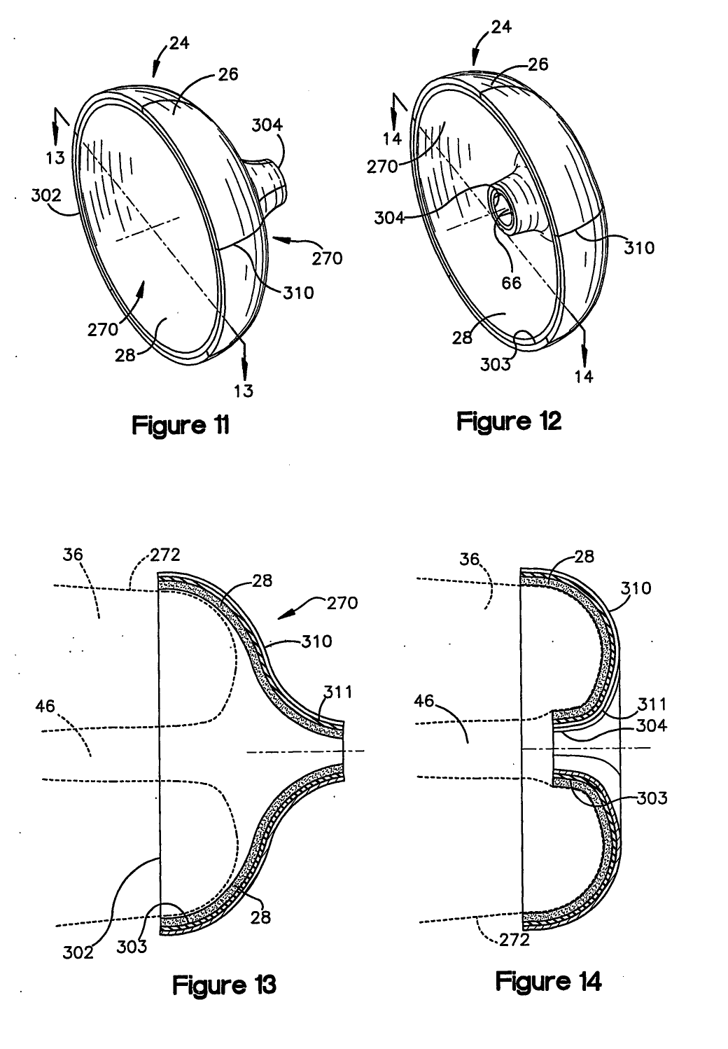

In the embodiments illustrated by FIGS. 11-16, retainers 270, 270′ are included that secure the barrier 26 to the cervix 36. A retainer may be used to assist the matrix in securing the drug delivery member to the cervix. In the alternative, a matrix with substantial adhesive properties is omitted and the retainer secures the barrier to the cervix. The retainers maintain the cap on the outer periphery of the cervix 36 and the projection in the cervical canal 46. The illustrated retainers 270, 270′ apply pressure to an outer circumferential surface 272 of the cervix and the cervical canal to secure the barrier to the cervix.

FIGS. 11-14 illustrate a retainer 270 that comprises ribs 310 that apply pressure to an outer circumferential surface of the cervix and the cervical canal to secure the barrier to the cervix (see FIG. 14). FIGS. 11 and 13 depict a barrier of one embodiment prior to insertion. The barrier 26 is funnel shaped before insertion, thus being tapered from a first end 302 to a second end 304. The first and second ends each have an opening, wherein the second end opening 304 is smaller than the first end opening 302. The barrier 26 may be comprised of any pliable or flexible material such as a pliable polymer, rubber or soft plastic. The matrix containing the medication is disposed on the barrier.

The interior surfaces of funnel-shaped opening 302 of the barrier 26 is sized for receiving a cervix therein. The interior walls 303 of the barrier are positioned for mating to the outer surface of the ectocervix so that therapeutic drugs contained within the matrix 28 may be applied to said surface. As will be described in detail below, the second opening 304 of the barrier becomes positioned within a patient's cervix after the barrier is placed over the cervix. The opening 304 allows the passage of fluids there through.

The barrier includes one or more support ribs 310 which are fabricated from a resilient material. The support ribs 310 may be comprised of a superelastic alloy or shape memory alloy such as a nickel titanium based alloy. Shape memory alloys undergo a transition between an austenitic state and a martensitic state at certain temperatures. When the ribs 310 are deformed while in the martensitic state the ribs retain this deformation as long as the ribs are retained in this state. The ribs revert to the original configuration when the shape memory alloy is heated to a transition temperature, at which time the ribs transform to the austenitic state. The temperatures at which these transitions occur are affected by the nature of the alloy and the type of material. More preferably, the shape memory alloy material has a transition temperature slightly lower than body temperature in order to enable a rapid transition of the ribs from the martensitic state to the austenitic state when the drug delivery member is implanted.

Referring to FIGS. 11 and 13, the barrier 26 has a funnel shape in the martensitic state prior to installation on the cervix. Referring to FIGS. 12 and 14, after installation on the cervix, the barrier 26 is heated by the warmer internal temperature of the patient until the shape of the drug delivery member causes the drug delivery member to attach to the cervix. Initially, the ribs 310 have a general “S” shape as illustrated by FIG. 13. Referring to FIG. 14, the shape memory alloy material of the ribs 310 transitions the ribs to the austenitic state and have a generally “C” cross sectional shape. As the ribs change shape, the opening 304 of the second end is pulled up into the opening of the first end, so that the interior surface of the cervix is in contact with the interior surface of the second end (see FIG. 14). As a result; the barrier is, maintained on the outer periphery of the cervix 36 and in the cervical canal. This allows the therapeutic drugs to be administered to the endocervix, and in particular, the squamo-columnar epithelial junction. As shown in FIG. 14, the lower portion 311 of rib 310 is thus in close proximity to the cervical lips, thereby functioning as a clip to hold the matrix in close contact with the cervix, thus allowing the therapeutic drugs to be administered thereto. The drug delivery member shown in FIGS. 11-14 may be installed onto a patient's cervix utilizing conventional instruments known in the art such as a cervical manipulator.

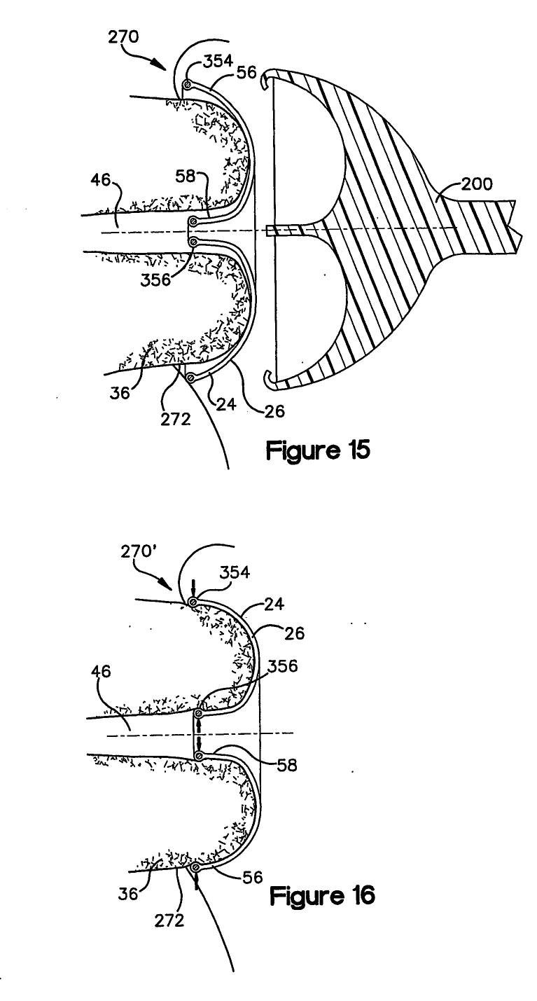

FIGS. 15 and 16 illustrate a drug delivery member of yet another embodiment that includes retainer 270′. The retainer 270′ applies pressure to an outer circumferential surface 272 of the cervix and the cervical canal 46 to secure the barrier to the cervix. In the embodiment illustrated by FIGS. 15 and 16, the barrier includes a cap portion 56 and a projection 58. The cap portion 56 is configured to fit over and approximately match the outer periphery 272 of a cervix. The projection 58 is radially inward of the cap portion and is configured to extend into the cervical canal 46. In the illustrated embodiment, the projection 58 extends past the squamo-columnar epithelial junction. The medication is applied to the matrix such that the medication is concentrated in the area surrounding the squamo-columnar epithelial junction when the drug delivery member is applied to a cervix. The retainer 270′ comprises an elastic band 354 secured to the cap and a ring 356 secured to the projection that expands upon insertion. The ring 356 may be made from a shape memory alloy.

FIGS. 15 and 16 schematically illustrate application of a drug delivery member 24 that includes retainer 270′ onto a patient's cervix. FIG. 15 shows the drug delivery member applied by the applicator 200 onto the cervix 36. The applicator shown in FIG. 16 holds the elastic band 354 in an expanded state until the drug delivery member is applied to the cervix. In the expanded state, the elastic band 354 can be easily placed over the cervix. The ring 356 is initially in an unexpanded state. In the unexpanded state, the ring 356 can be easily inserted into the cervical canal. Referring to FIG. 16, the ring 356 expands a short time after insertion and secures the projection 352 in the cervical canal 46. The applicator 200 is removed and the elastic band 354 secures the drug delivery member to the outer periphery of the cervix.

While the invention has been described with reference to specific embodiments, it will be apparent to those skilled in the art that may alternatives, modifications, and variations may be made. Accordingly, the present invention is intended to embrace all such alternatives, modifications, and variations that may fall within the spirit and scope of the appended claims.

I claim:

1. A drug delivery member for applying medication to internal human tissue, comprising:
a) a medication impermeable barrier shaped to conform to a contour of the internal tissue;
b) a medication carrying matrix disposed on the barrier, the matrix being adapted to adhere to the internal human tissue and release the medication to the human tissue upon being applied to the internal human tissue.

2. The drug delivery member of claim 1, further comprising a medication-free deposit of matrix in a region near an edge of the barrier that inhibits migration of the medication past the edge of the barrier.

3. The drug delivery member of claim 1, wherein the barrier is formed from a pliable material that conforms to a surface of the internal human tissue.

4. The drug delivery member of claim 1, wherein the barrier is formed with an inner surface that corresponds to an outer surface of a cervix.

5. The drug delivery member of claim 1, wherein the barrier includes a cap portion that is configured to fit over an outer periphery of a cervix and includes a projection radially inward of the cap portion that is configured to extend into the cervical canal.

6. The drug delivery member of claim 5, wherein the projection is sized to extend past a squamo-columnar epithelial junction.

7. The drug delivery member of claim 6 wherein the medication is dispersed in the matrix such that the medication is concentrated in an area surrounding the squamo-columnar epithelial junction when the drug delivery member is applied to a cervix.

8. The drug delivery member of claim 1 wherein the medication is a drug selected from the group consisting of: 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, or subcombinations thereof.

9. The drug delivery member of claim 1, wherein the medication is 5-fluorouracil or cis-platinum.

10. The drug delivery member of claim 1, wherein the matrix is a synthetic carboxy polymer, a natural carboxy polymer, or carboxy co-polymers whose fluidity is pH dependent.

11. The drug delivery member of claim 1, further comprising a string attached to the barrier for removing the drug delivery member.

12. The drug delivery member of claim 1, further comprising a retainer for securing the drug delivery member to the internal tissue.

13. The drug delivery member of claim 1, wherein the barrier dissolves after the medication is transferred from the matrix to the internal tissue.

14. A method for inserting a drug delivery member onto the cervix of a patient comprising the steps of: mounting a drug delivery member onto an applicator; inserting the applicator near the cervix of said patient; and releasing said drug delivery member from said applicator onto the cervix of the patient.

15. The method of claim 14, further comprising the step of inflating a portion of said applicator.

16. The method of claim 14, wherein mounting the drug delivery member on the applicator comprises retaining an elastic band of said drug delivery member on the applicator.

17. The method of claim 14, wherein said drug delivery member further comprises a drug selected from the group consisting of: 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, or subcombinations thereof.

18. The method of claim 14, wherein a first portion of the drug delivery member is placed over an outer periphery of a cervix and a second portion of the drug delivery member is inserted into the cervical canal.

19. The method of claim 18, wherein the second portion is inserted past a squamo-columnar epithelial junction.

20. The method of claim 19, wherein the medication is applied to the drug delivery member such that the medication is concentrated in an area surrounding the squamo-columnar epithelial junction when the drug delivery member is applied to a cervix.

21. A method for treating cervical intraepithelial neoplasia comprising the steps of: mounting a drug delivery drug delivery member containing a drug selected from the group consisting essentially of: 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, and subcombinations thereof onto an applicator; inserting the applicator near the cervix of said patient; and releasing said drug delivery member from said applicator onto the cervix of-the patient.

22. The method of claim 21, further comprising the step of inflating a portion of said applicator.

23. The method of claim 21, further comprising the step of retaining said drug delivery member onto said applicator prior to insertion onto the cervix of the patient.

24. The method of claim 21, wherein a first portion of the drug delivery member is placed over an outer periphery of a cervix and a second portion of the drug delivery member is inserted into the cervical canal.

25. The method of claim 24, wherein the second portion is inserted past a squamo-columnar epithelial junction.

26. The method of claim 25, wherein the medication is applied to the drug delivery member such that the medication is concentrated in an area surrounding the squamo-columnar epithelial junction when the drug delivery member is applied to a cervix.

27. A drug delivery member for administering medication to the cervix of a patient, comprising:
a) a medication impermeable barrier including a cap that is configured to fit over an outer periphery of the cervix and a projection radially inward of the cap portion that is configured to extend into a cervical canal;
b) a medication carrying matrix disposed on the barrier that releases the medication to the cervix when the barrier is secured on the cervix;
c) a retainer that secures the barrier to the cervix, such that the cap is maintained on the outer periphery of the cervix and the projection is maintained in the cervical canal.

28. The drug delivery member of claim 27, wherein the retainer applies pressure to an outer circumferential surface of the cervix and the cervical canal to secure the barrier to the cervix.

29. The drug delivery member of claim 27, wherein the retainer comprises one or more ribs that apply pressure of an outer circumferential surface of the cervix and the cervical canal to secure the barrier to the cervix.

30. The drug delivery member of claim 27, wherein the retainer comprises an elastic band secured to the cap and a ring secured to the projection that expands upon insertion.

31. The drug delivery member of claim 27, further comprising a medication-free deposit of matrix in a region near an edge of the barrier such that the medication free deposit inhibits migration of the medication past the edge of the barrier.

32. The drug delivery member of claim 27, wherein the projection is sized to extend past a squamo-columnar epithelial junction.

33. The drug delivery member of claim 32, wherein the medication is dispersed in the matrix such that the medication is concentrated in an area surrounding the squamo-columnar epithelial junction when the drug delivery member is applied to a cervix.

34. The drug delivery member of claim 27, wherein the medication is a drug selected from the group consisting of: 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, and subcombinations thereof.

35. The drug delivery member of claim 27, wherein the matrix is a synthetic carboxy polymer, a natural carboxy polymer, or carboxy co-polymers whose fluidity is pH dependent.

36. A drug delivery member for administering therapeutic agents to the cervix of a patient, the drug delivery member comprising:
a) a tubular member having a first opening and a second opening, wherein the first opening is larger than the second opening; said tubular member being formed of a flexible material containing a therapeutic agent; and
b) one or more ribs connected to the tubular member, said ribs being selectively deformable between a first state where the ribs allow the tubular member to be placed over the cervix and a second state where the ribs secure the tubular member to the cervix.

37. The drug delivery member of claim 36, wherein the one or more ribs are comprised of a shape memory alloy.

38. The drug delivery member of claim 36, wherein the tubular member is comprised of a bioadhesive material.

39. The drug delivery member of claim 38, wherein the bioadhesive material is a synthetic carboxy polymer, a natural carboxy polymer, or carboxy co-polymers whose fluidity is pH dependent.

40. The drug delivery member of claim 36, wherein the tubular member is comprised of a bioadhesive material selected from the group consisting of: carbopol 934, pectin, polyvinylpyrolidon, guar gum, ethylene maleic anhydride resins and mixtures thereof.

41. The drug delivery member of claim 36, wherein the rib is comprised of a nickel titanium alloy.

42. The drug delivery member of claim 36, wherein the therapeutic agent is selected from the group consisting of: 5-fluorouracil, cis-platinum, trans-retinoic acid, 4-hydroxyphenylretinamide, Imiquimod, betacarotine, dihematoporphyrin ether, cidofovir (Vistide), 5-aminolevulinic acid, recombinant human beta interferons, alpha-difluoromethylornithine, ifosfamide, leucovorin, idoxuridine and acrarubicin, and subcombinations thereof.
